(19) United States
(12) Patent Application Publication
(10) Pub. No.: US 2017/0114314 A1
(43) Pub. Date: Apr. 27, 2017

(54) SKIN EXPANSION APPARATUS AND SKIN EXPANSION METHOD USING THE SAME

(71) Applicants: ELECTRONICS AND TELECOMMUNICATIONS RESEARCH INSTITUTE, Daejeon (KR); KYUNGPOOK NATIONAL UNIVERSITY HOSPITAL, Daegu (KR)

(72) Inventors: Hwi Gang KIM, Daegu (KR); Yun Jeong SONG, Daejeon (KR); Chang Hyuk HONG, Daegu (KR); Sun Hee AN, Gyeongsan (KR); Jeong Ok LIM, Daegu (KR); Dae Sik KIM, Daegu (KR); Hyung Soo LEE, Daegu (KR); Eun Chang CHOI, Daegu (KR)

(21) Appl. No.: 15/229,031

(22) Filed: Aug. 4, 2016

(30) Foreign Application Priority Data
Oct. 27, 2015 (KR) 10-2015-0149569

(51) Int. Cl. C12M 1/42; C12M 3/00; C12N 5/071

(52) U.S. Cl. CPC C12M 35/04; C12N 5/0698; C12M 21/08

(57) ABSTRACT

A skin expansion apparatus comprises a chamber accommodating a culture medium in which a skin piece is put, and at least two clamps to fix the skin piece. The clamps pull the skin piece in an outward direction to apply tensile force to the skin piece in a state in which the skin piece is put in the culture medium.

### CROSS-REFERENCE TO RELATED APPLICATION

The present application claims priority to Korean patent application number 10-2015-0149569 filed on Oct. 27, 2015 the entire disclosure of which is incorporated herein in its entirety by reference.

### BACKGROUND

1. Field

An embodiment of the present invention relates to a skin expansion apparatus for expanding external skin, and a skin expansion method using the same.

2. Description of the Related Art

Skin is one of human organs, which represents about 16% of an overall weight of a human being. Skin is in direct contact with an external environment and serves to protect a human being from the outside. For example, skin acts as anatomical barrier with respect to a material such as pathogenic bacterium present outside. Also, skin serves to prevent essential nutrient from being washed out from a body, and provides a semi-transparent barrier preventing an excessive amount of secretion loss. Other functions of skin include insulation, temperature control, and a sensory action.

Skin may be damaged by burn, injury, or depigmentation (for example, leukoplakia), and in this case, damaged skin may be recovered through transplantation.

### SUMMARY

An embodiment of the present invention relates to a skin expansion apparatus capable of expanding and culturing skin collected for transplantation.

Another embodiment of the present invention relates to a method for expanding skin by using the skin expansion apparatus.

A skin expansion apparatus according to an embodiment of the present invention includes a chamber accommodating a culture medium in which a skin piece is put; and at least two clamps to fix the skin piece. The clamps face each other in the chamber. The clamps pull the skin piece in an outward direction to apply tensile force to the skin piece in a state in which the skin piece is put in the culture medium.

The clamps may include first clamps provided on both sides of the skin piece in a first direction.

The clamps may include second clamps provided on both sides of the skin piece in a second direction, and the second direction may cross the first direction.

Each of the clamps may include an upper plate to contact with an upper surface of the skin piece, a lower plate to contact with a lower surface of the skin piece, and at least one needle protruding from at least any one of the upper plate and the lower plate and penetrating through the skin piece.

The skin expansion apparatus may further include: a clamp module provided in the chamber and detachably attached; and a clamp connection bar connected to the clamp module and each of the clamps.

The skin expansion apparatus may further include a controller controlling an expansion direction of the skin piece and an expansion time of the skin piece by changing a position of at least one of the clamp module and the clamp connection bar.

The skin expansion apparatus may further include an environment controller controlling a temperature of an interior of the chamber.

The chamber may have a bottom part and a side wall part connected to the bottom part, and the environment controller may include a water channel in which warm water moves within the side wall part. The environment controller may further include an inlet to provide the warm water to the water channel and an outlet to drain the warm water from the water channel.

At least one of the inlet and the outlet may further include a valve.

A method for expanding skin by using the skin expansion apparatus according to another embodiment of the present invention includes: a skin fixing step of fixing a skin piece within a chamber accommodating a culture medium by using clamps; and a skin expanding step of pulling the skin piece in an outward direction by using the clamps in a state in which the culture medium is put.

The skin expansion apparatus may further include: a operating controller controlling an expansion direction of the skin piece and an expansion time of the skin piece, and the skin expanding step may be performed in one of a manual mode in which an expansion time of the skin piece is selected by a user and an automatic mode in which an expansion time of the skin piece is selected according to a preset value.

The skin expansion apparatus may include a clamp module provided in the chamber and detachably attached and a clamp connection bar connected to the clamp module and each of the clamps, and when the manual mode and the automatic mode are selected, positions of the clamp module and the clamp connection bar may be changed.

The method may further include: replacing the culture medium after the skin expanding step.

A temperature of the chamber may be maintained as a temperature of a human body when the skin expanding step is performed.

In the skin expansion apparatus according to the embodiment of the present invention, an area of skin is expanded by stimulating skin by pulling skin using tensile force. The skin expansion apparatus according to the embodiment of the present invention stably expands and cultures skin piece having a small area collected from a doner part with a predetermined area or greater, when transplantation is performed. The skin expansion apparatus according to the embodiment of the present invention expands and cultures the skin piece without rupture and prevents skin necrosis to the maximum.

### BRIEF DESCRIPTION OF THE DRAWINGS

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will full convey the scope of the example embodiments to those skilled in the art.

In the drawing figures, dimensions may be exaggerated for clarity of illustration. It will be understood that when an element is referred to as being “between” two elements, it can be the only element between the two elements, or one or more intervening elements may also be present. Like reference numerals refer to like elements throughout.

FIG. 1 is a perspective view illustrating a skin expansion apparatus according to an embodiment of the present invention;

FIG. 2A is a cross-sectional view illustrating a clamp according to an embodiment of the present invention, FIG. 2B is a plan view when an upper plate of FIG. 2A is viewed from below, and FIG. 2C is a plan view when a lower plate of FIG. 2A is viewed from above;

FIG. 3 is a plan view illustrating a portion of a skin expansion apparatus according to the embodiment of the present invention of FIG. 1, illustrating only a skin piece as a target to be expanded and clamps of a skin expansion apparatus of the present invention; and

FIGS. 4 to 6 are plan views illustrating skin expansion apparatuses according to embodiments of the present invention, illustrating only a skin piece as a target to be expanded and clamps of a skin expansion apparatus of the present invention.

### DETAILED DESCRIPTION

The present invention may be modified variably and may have various embodiments, particular examples of which will be illustrated in drawings and described in detail. However, it is to be understood that the present invention is not limited to a specific disclosed form, but includes all modifications, equivalents, and substitutions without departing from the scope and spirit of the present invention.

In describing drawings, like numbers refer to like elements. In the accompanying drawings, dimensions of structures are exaggerated to clarify the present invention. While terms such as “first” and “second,” etc., may be used to describe various components, such components must not be understood as being limited to the above terms. The above terms are used only to distinguish one component from another. For example, a first component may be referred to as a second component without departing from the scope of rights of the present invention, and likewise a second component may be referred to as a first component. As used herein, the singular forms “a”, “an” and “the” are intended to include the plural forms as well, unless the context clearly indicates otherwise.

In the present application, it is to be understood that the terms such as “including” or “having,” etc., are intended to indicate the existence of the features, numbers, operations, actions, components, parts, or combinations thereof disclosed in the specification, and are not intended to preclude the possibility that one or more other features, numbers, operations, actions, components, parts, or combinations thereof may exist or may be added. Also, it will be understood that when an element such as a layer, film, region, or substrate is referred to as being “on” another element, it can be directly on the other element or intervening elements may also be present. Conversely, it will be understood that when an element such as a layer, film, region, or substrate is referred to as being “beneath” another element, it can be directly on the other element or intervening elements may also be present.

An embodiment of the present invention includes an apparatus for expanding a collected skin piece. The skin expansion apparatus is an apparatus for expanding a skin piece having a small area so as to be transplanted to a damaged part having a large area.

Hereinafter, an embodiment of the present invention will be described with reference to the accompanying drawings.

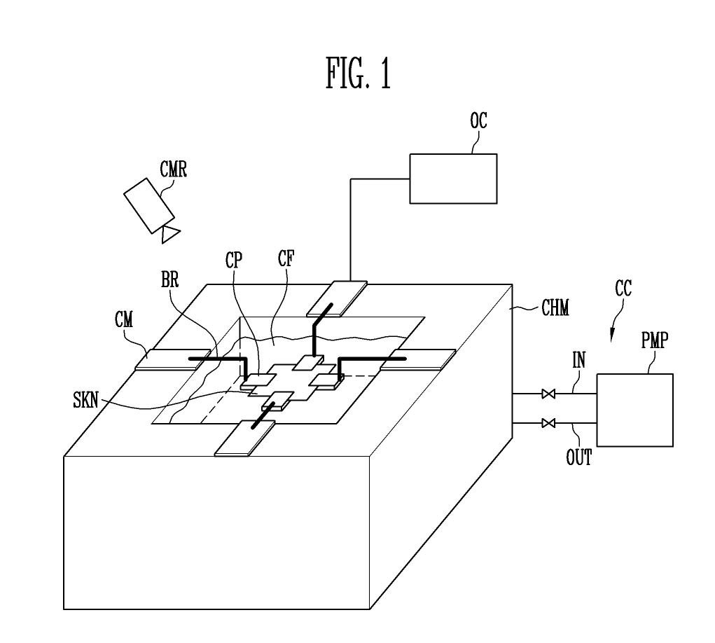

FIG. 1 is a perspective view illustrating a skin expansion apparatus according to an embodiment of the present invention.

Referring to FIG. 1, a skin expansion apparatus according to an embodiment of the present invention comprises a chamber CHM accommodating a culture medium CF in which a skin piece SKN is put, an environment controller CC controlling an environment of an interior of the chamber CHM, a clamp unit for fixing the skin piece SKN, and a operating controller OC operating the clamp unit.

The skin piece SKN is separated from a skin of a human body and is to recover damaged skin. The skin piece SKN may be obtained by excising a part of own skin or by excising a part of skin of a different person. The skin piece SKN may be used to be transplanted to a skin-damaged patient.

The skin piece SKN may be obtained through various known schemes. For example, the skin piece SKN may be obtained by marking an area degree of part to be given, and cutting skin of the marked part.

The chamber CHM provides a space in which the skin piece SKN is expanded. The chamber CHM may provide a space opened in an upward direction, and, in this case, the chamber CHM may include a bottom part and a side wall part upwardly extending from the bottom part.

The culture medium CF for culturing the skin is provided in the space. The skin piece SKN is put in the culture medium CF, and while the skin piece SKN is being expanded, the skin piece is also cultured. The culture medium CF may be added or exchanged with a new culture medium as necessary while the expansion is being performed or after the expansion is performed.

The culture medium CF, which is to culture the skin piece SKN, may include macroelements and microelements required for grown cells of the skin piece SKN. A known composition of the culture medium CN may be used without being limited.

The environment controller CC controls an environment within the chamber, in particular, an environment related to a temperature.

Preferably, the culture medium CF is maintained at a temperature at which the skin piece SKN is easily cultured. The environment controller CC may include a heater for transmitting heat to the chamber CHM, and transmits heat to the culture medium CF so that the culture medium CF may maintain a predetermined temperature, for example, the same temperature as that of a human body.

According to an embodiment of the present invention, the environment controller CC may include a heater using warm water. In this case, the environment controller CC may include a water channel (not shown) provided within the side wall part of the chamber CHM and allowing warm water to move therethrough, an inlet IN to provide warm water to the water channel, and an outlet OUT to drain warm water from the water channel.

The water channel may be provided in the form of a void within the side wall part. In this case, the side wall part may have a structure of a dual-wall having an inner wall and an outer wall. However, a shape of the water channel within the side wall part is not limited thereto and the water channel may have various other shapes. For example, the water channel may be provided in the form of a tube or a hose and may be bent a plurality of times.

The inlet IN is connected to one side of the water channel and provides warm water to the water channel from the outside. A valve may be installed in the inlet IN, an amount of warm water provided through the inlet IN or a speed for providing warm water may be adjusted through the inlet IN.

The outlet OUT is connected to the other side of the water channel, and discharges the warm water to the outside from the water channel A valve may be installed in the outlet OUT, and an amount of warm water discharged through the outlet OUT or a speed for discharging warm water may be adjusted using the valve.

According to an embodiment of the present invention, a circulation pump PMP may be connected to one side of the inlet IN. The circulation pump PMP may pump warm water toward the inlet IN. According to an embodiment of the present invention, the circulation pump PMP may also be connected to the outlet OUT, and in this case, the circulation pump PMP heats water discharged to the outlet OUT again and provides the heated water toward the inlet IN.

The environment controller CC using warm water provides warm water to the inlet IN through the circulation pump PMP, and warm water provided to the inlet IN is moved along the water channel within the side wall of the chamber CHM and discharged to the outlet OUT. The warm water transmits heat to the culture medium CF of the chamber CHM, while moving along the water channel. Warm water discharged to the outlet OUT may be provided again to the inlet IN through the circulation pump PMP.

Although not shown, a heating member for heating warm water may be provided on the side of the circulation pump PMP.

According to an embodiment of the present invention, a type of the environment controller CC is not limited thereto, and the environment controller CC may have any other type as long as it can control a temperature environment of the chamber CHM. For example, the environment controller CC may be provided in the form of a heating coil provided within the side wall part of the chamber CHM.

The clamp unit is to expand the skin piece SKN in a state in which the culture medium CF is put. The clamp unit pulls the skin piece SKN in an outward direction to apply tensile force to the skin piece SKN.

The clamp unit may include clamps CP fixing the skin piece SKN, a clamp connection bar BR connected to the clamps CP, and a clamp module CM connecting the clamp connection bar BR and the chamber CHM.

The clamps CP are provided in a plural, and directly grip the skin piece SKN. For example, the clamps CP are provided as at least two clamps in order to grip both ends of the skin piece SKN facing each other. The clamps CP grip both ends of the skin piece SKN and pull the skin piece SKN in an outward direction to apply tensile force.

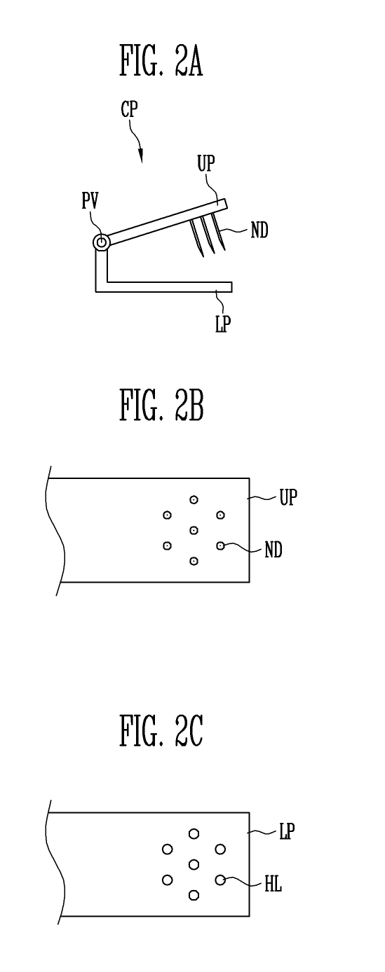

FIG. 2A is a cross-sectional view illustrating a clamp according to an embodiment of the present invention, FIG. 2B is a plan view when an upper plate of FIG. 2A is viewed from below, and FIG. 2C is a plan view when a lower plate of FIG. 2A is viewed from above.

Referring to FIGS. 1 and 2A to 2C, each of the clamps CP may include an upper plate UP in contact with an upper surface of the skin piece SKN, a lower plate LP in contact with a lower surface of the skin piece SKN, and at least one needle ND protruding from at least one of the upper plate UP and the lower plate LP and penetrating through the skin piece SKN.

The upper plate UP and the lower plate LP are connected through a pivot PV, and at least one of the upper plate UP and the lower plate LP may rotatably move in a partial degree, centered around the pivot PV.

In the clamps CP of the present invention, the upper plate UP and the lower plate LP are open such that the skin piece SKN may enter, and may be sufficiently pressed so as to allow the needle ND to sufficiently enter the skin after the skin piece SKN is put. That is, the skin piece SKN is disposed between the upper plate UP and the lower plate LP and may be fixed by the needle ND. When the skin piece SKN is gripped by the clamp CP, the upper surface of the upper plate UP may be directly in contact with the upper surface of the skin piece SKN and the upper surface of the lower plate LP may be directly in contact with the lower surface of the skin piece SKN.

The needle ND penetrates through the skin piece SKN to firmly fix the skin piece SKN between the upper plate UP and the lower plate LP. In an embodiment of the present invention, it is illustrated that the needle ND protrudes in a direction from the upper plate UP to the lower plate LP, but the present invention is not limited thereto and the needle ND may protrude in a direction from the lower plate LP to the upper plate UP.

In an embodiment of the present invention, damage to the gripped skin piece SKN is minimized by using the needle ND in the clamp CP. The needle ND is applied to a very small portion of the area of the skin piece SKN, and since the needle ND is stuck into the skin piece SKN in a direction vertical to tensile force applied to the skin piece SKN, damage to the skin in the stage in which the skin piece SKN expands is minimized. Also, the skin piece SKN is not easily detached from the clamp CP due to the needle ND.

In an embodiment of the present invention, the needle ND may be provided as one or a plurality of needles. When the needle ND is provided as a plurality of needles, the needles ND may be arranged in various forms in order to minimize damage to the skin piece SKN and effectively grip the skin piece SKN. For example, the plurality of needles ND may be arranged in a zigzag form so as to go crisscross to each other in a direction in which the skin piece SKN is pulled. This is to prevent damage that may occur as tensile force applied to the skin piece SKN concentrates on a specific portion.

A guide hole HL may be provided in a portion of the lower plate LP corresponding to a position in which the needle ND is provided. The needled ND enters the guide hole HL when the clamp is closed. When the clamp is closed, the guide hole HL guides a path along which the needle ND moves, and prevents wearing of an end portion of the needle ND.

In an embodiment of the present invention, shapes of the upper plate UP, the lower plate LP, the needle ND, and the pivot PV of the clamp CP may be variably modified within a limit not exceeding the principle of fixing the skin piece SKN.

The clamp connection bar BR is provided in a bar shape in which one end is connected to the clamp CP and the other end is connected to the clamp module CM. The clamp connection bar BR may be bent a plurality of times as necessary. The clamp connection bar BR may have a plurality of connection columns connected to each other in the bend portion, and in this case, the plurality of connection columns may be fastened to each other in the bend portion. Thus, each of the bent and separated portions of the clamp connection bar BR may be adjusted in length and position, and mutually adjacent separated portions may be fastened. For example, a pivot PV maybe provided in the bent portion of the clamp connection bar BR. The clamp connection bar BR may be driven by the operating controller OC and position the skin piece SKN as close as possible to the bottom portion of the chamber CHM. In this case, a large amount of culture medium CF is not necessary, and thus, a usage amount of the culture medium CF may be minimized.

The clamp module CM is connected to the clamp connection bar BR and provided on an upper portion of the chamber CHM. In detail, the clamp module CM may be provided on an upper portion of a side wall of the chamber CHM. The clamp module CM may include a magnet (not shown), and may be attached to or detached from the chamber CHM using magnetic force of the magnet.

The operating controller OC controls operating of the clamps, and controls an expansion direction of the skin piece SKN and an expansion time of the skin piece SKN by changing a position of at least one of the clamp module CM and the clamp connection bar BR.

For example, the operating controller OC may move the clamp module CM in at least one of directions (that is, a horizontal direction) vertical to gravity or may move the clamp connection bar BR in a direction parallel to gravity and/or in a direction vertical to gravity. In an embodiment of the present invention, the operating controller OC may control the clamp module CM to move forward or backward along one direction. The operating controller OC may have a cylinder type sliding structure for changing positions of the clamp module CM and the clamp connection bar BR.

In an embodiment of the present invention, the clamp module CM may be moved in a predetermined direction (for example, a horizontal direction) on the side wall of the chamber CHM in consideration of an expansion direction of the skin piece SKN. The clamp connection bar BR may also be moved in a predetermined direction (for example, a horizontal direction and a vertical direction) in a portion linking the clamp module CM and the clamps CP in consideration of the expansion direction of the skin piece SKN.

According to an embodiment of the present disclosure, the skin expansion apparatus may further include a camera unit CMR. The camera unit CMR observes, records, or captures whether the skin piece SKN is expanded or cultured. In an embodiment of the present invention, a state of the skin piece SKN may be checked through the camera unit CMR, and whether to additionally expand or culture the skin piece SKN may be determined.

Since the skin expansion apparatus having the foregoing configuration expands the skin piece SKN and simultaneously cultures the skin piece SKN by using the clamp unit, a skin piece SKN having an area larger than that of the first collected skin piece SKN may be acquired.

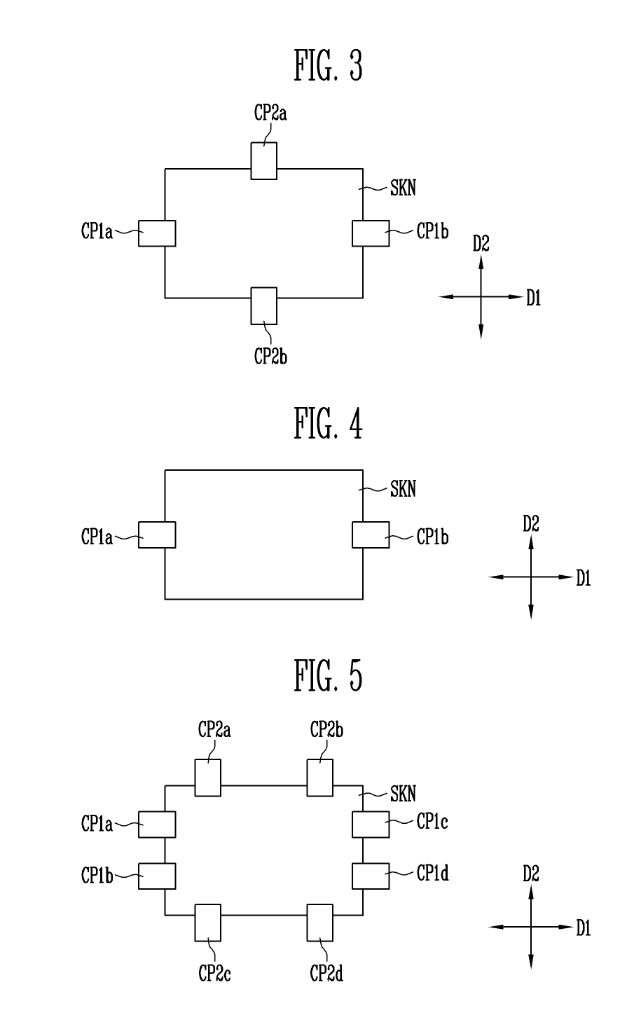

FIG. 3 is a plan view illustrating a portion of a skin expansion apparatus according to the embodiment of the present invention of FIG. 1, illustrating only a skin piece as a target to be expanded and clamps of a skin expansion apparatus of the present invention.

Referring to FIG. 3, the skin piece SKN desired to be expanded is fixed by clamps CP1a, CP1b, CP2a, and CP2b.

In the embodiment, the skin piece SKN is provided as having a rectangular shape, but the present invention is not limited thereto and the skin piece SKN may have various shapes (for example, polygonal shapes including circular, oval, semicircular, and triangular shapes).

The clamps CP1a, CP1b, CP2a, and CP2b include first clamps CP1a and CP1b disposed at both ends of the skin piece SKN in a first direction D1 and second clamps CP2a and CP2b disposed at both ends of the skin piece SKN in a second direction D2. The second direction D2 may be a direction different from the first direction D1 and may be a direction vertically crossing the first direction D1.

The first clamps CP1a and CP1b face each other and pull the skin piece SKN along the first direction D1 outwardly to apply tensile force to the skin piece SKN in the first direction D1. Here, the first clamps CP1a and CP1b facing each other may have the same shape. Since the skin piece SKN is in a state of being put in the culture medium, the skin piece SKN expands along both ends of the first direction D1, as culturing is performed.

The second clamps CP2a and CP2b face each other and pull the skin piece SKN along the second direction D2 outwardly to apply tensile force to the skin piece SKN in the second direction D2. Here, the second clamps CP2a and CP2b facing each other may have the same shape. Since the skin piece SKN is in a state of being put in the culture medium, the skin piece SKN expands along both ends of the second direction D2, as culturing is performed.

As described above, it is possible to obtain the skin piece SKN having an area larger than the area when first collected by expanding the skin piece SKN in the first direction D1 and the second direction D2 by using the first clamps CP1a and CP2a and the second clamps CP2a and CP2b.

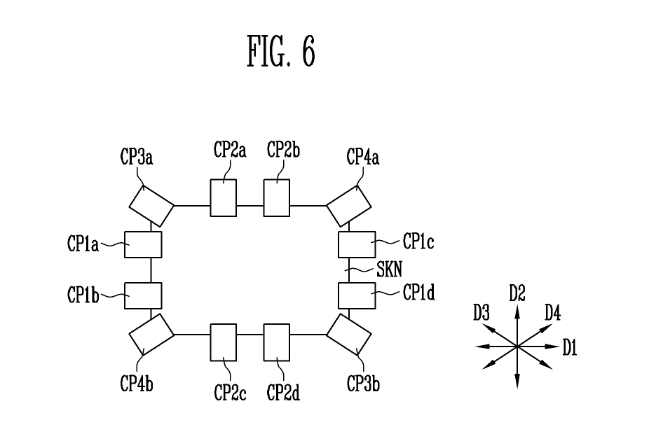

The numbers and positions of the first clamps CP1a and CP2a and the second clamps CP2a and CP2b may be differently set according to a shape of the skin piece SKN or direction in which the skin piece SKN is desired to be expanded. This will be described with reference to FIGS. 4 and 5.

FIGS. 4 to 6 are plan views illustrating skin expansion apparatuses according to embodiments of the present invention, illustrating only a skin piece as a target to be expanded and clamps of a skin expansion apparatus of the present invention.

Referring to FIG. 4, in the skin expansion apparatus according to an embodiment of the present invention, the skin piece SKN is fixed by the first clamps CP1a and CP2a positioned at both ends of the skin piece SKN in the first direction D1. In the present embodiment, the skin piece SKN may be expanded to outside of both ends of the skin piece SKN in the first direction D1 by the first clamps CP1a and CP2a and may not be expanded in a direction other than the first direction D1.

Referring to FIG. 5, in the skin expansion apparatus according to an embodiment of the present invention, the skin piece SKN may include first clamps CP1a, CP1b, CP1c, and CP1d disposed as a pair at one end of the skin piece SKN and another pair at the other end of the skin piece SKN in the first direction D1 and second clamps CP2a, CP2b, CP2c, and CP2d disposed as a pair at one end of the skin piece SKN and another pair at the other end of the skin piece SKN in the second direction D2. Here, the pairs of clamps (that is, CP1a and CP1c, CP1b and CP1d, CP2a and CP2c, and CP2b and CP2d) facing each other with the skin piece SKN interposed therebetween pull the skin piece SKN at different positions of the skin piece SKN outwardly, thus effectively expanding the skin piece SKN in the first and second directions D1 and D2.

Referring to FIG. 6, in the skin expansion apparatus according to an embodiment of the present invention, the skin piece SKN may include third clamps CP3a and CP3b positioned at both ends of the skin piece SKN in a third direction D3 crossing the first direction D1 and the second direction D2 and fourth clamps CP4a and CP4b positioned at both ends of the skin piece SKN in a fourth direction D4 crossing each of the first direction D1 and the second direction D2 and also crossing the third direction D2, in addition to the first clamps CP1a, CP1b, CP1c, and CP1d disposed as a pair at one end of the skin piece SKN and another pair at the other end of the skin piece SKN in the first direction D1 and the second clamps CP2a, CP2b, CP2c, and CP2d disposed as a pair at one end of the skin piece SKN and another pair at the other end of the skin piece SKN in the second direction D2. In this case, the skin piece SKN may be more easily expanded by the first to fourth clamps applying tensile force to the skin piece SKN in different directions.

Referring to FIGS. 3 to 6, the clamps may be provided in various directions and in various numbers according to a direction in which the skin piece is to be expanded. In other words, tensile force may be applied to the skin piece in a single direction along a single axis as illustrated in FIG. 4, and tensile force may be applied to the skin piece in multiple directions along multiple axes as illustrated in FIGS. 3, 5, and 6.

A method for expanding skin by using the skin expansion apparatus as described above includes preparing a skin piece, a skin fixing step of fixing the skin piece to an interior of a chamber in which a culture medium is accommodated, and a skin expanding step of pulling the skin piece in an outward direction by using the clamps in a state in which the culture medium is put. This will be described sequentially as follows.

First, a skin piece SKN as a target to be expanded is prepared.

The skin piece SKN may be obtained through various known methods. For example, the skin piece SKN may be obtained by marking an area degree of part to be given, and cutting skin of the marked part.

The skin piece SKN may be provided as having a rectangular shape, but the present invention is not limited thereto and the skin piece SKN may have various shapes (for example, polygonal shapes including circular, oval, semicircular, and triangular shapes).

Next, the skin piece is fixed by clamps and disposed within a chamber in which a culture medium is accommodated. The culture medium may be maintained at a temperature at which culturing is easily performed by an environment controller. The temperature at which culturing is easy may be variously set according to the skin piece and is set to a temperature similar to or the same as a condition within a body at the maximum. In an embodiment of the present invention, when the skin piece stems from a human body, the culture medium may be maintained to have a body temperature (about 36.5° C.) so that the piece may be sufficiently cultured even at outside of a body.

Thereafter, the skin piece is pulled for a predetermined time in an outward direction of a direction in which the skin piece is to be expanded by using the clamps to apply tensile force to the skin piece.

In the embodiment, in the step of expanding the skin piece, an expansion direction of the skin piece and an expansion time of the skin piece may be controlled by a operating controller. The operating controller may control the skin expansion direction and the skin expansion time by adjusting positions of the clamp module, the clamp connection bar, and the clamps according to a user selection.

Here, the user may select any one of a manual mode and an automatic mode with respect to the expansion direction of the skin piece and the expansion time of the skin piece.

When selecting the manual mode, the user may set and input an expansion direction of the skin piece and an expansion time of the skin piece. The skin piece is expanded according to the input matter.

When the automatic mode is selected, the skin piece is expanded according to a preset expansion time of the skin piece and a preset expansion time of the skin piece. The preset expansion direction of the skin piece and the preset expansion time of the skin piece may be provided as a plurality of selections in advance.

In the step of expanding the skin piece, a degree of expanding and culturing the skin piece may be measured. After the skin piece is expanded, the culture medium may be replaced or added in consideration of a measurement value.

The skin piece expanded according to the method described above may be transplanted to a benefit part through transplantation. The transplantation is an operation of collecting a skin piece from a different part and transplanting the collected skin piece to an operation portion and suturing it when there is loss in a skin tissue that cannot be closed through first suturing. The transplantation may be performed to reconstruct a soft tissue in case of correction of scar and contracture due to injury such as burn, skin tumor removal and skin ulcers, or fingertip replantation.

According to an embodiment of the present invention, it is possible to effectively perform transplantation by collecting a skin piece having a small area and easily expanding the skin piece to have a large area by using the apparatus and method described above.

In detail, an existing skin transplantation is classified into artificial skin graft, graft using an internal tissue expander, and net type skin grating. The artificial skin graft is an operation of grating artificially synthesized or cultured substitute skin. The artificial skin graft accompanies different self-tissue graft and has limitations such as insufficient physical characteristics, high cost, absence of permanency, vulnerability to infection, or inadequate bio-suitability. Also, in the graft using an internal tissue expander, a skin expander is put into a body to inflate and expand a tissue, and the expanded tissue is used in an operation. The graft using an internal tissue expander has an advantage of using a self-tissue, but a great amount of time is consumed to secure a sufficient tissue and it is impossible to apply the graft using an internal tissue expander to an inadequate body position due to an obstacle in a daily life of a patient and fiberization of a tissue. Also, the net type skin grating is a method of grafting by expanding a skin graft piece by using a device like a net and grating the expanded skin piece. The net type skin grating has a very long injury recovery time, degrades skin scar and contracture, and requires a large amount of self-skin.

In comparison, the skin expansion apparatus and the skin expansion method according to an embodiment of the present invention minimize damage and scar of a doner part, and thus, a risk of immunity suitability and infection may be minimized. Also, in this connection, since a skin piece having a small area collected from the doner part is pulled by a predetermined force or stronger outside of a body so as to be expanded and cultured to have an area suitable for a benefit part, thus facilitating transplantation. In addition, in the skin expansion apparatus and the skin expansion method according to an embodiment of the present invention, the interior of a culture medium pulling a skin piece has an advantage of having an environment in which necrosis of skin is minimized and a skin tissue is maintained to be alive and grown.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated.

Accordingly, it will be understood by those of skill in the art that various changes in form and details may be made without departing from the spirit and scope of the present invention as set forth in the following claims.

What is claimed is:

1. A skin expansion apparatus comprising:
a chamber accommodating a culture medium in which a skin piece is put; and
at least two clamps to fix the skin piece, the clamp facing each other in the chamber,
wherein the clamps pull the skin piece in an outward direction to apply tensile force to the skin piece in a state in which the skin piece is put in the culture medium.

2. The skin expansion apparatus of claim 1, wherein the clamps include first clamps provided on both sides of the skin piece in a first direction.

3. The skin expansion apparatus of claim 2, wherein the clamp include second clamps provided on both sides of the skin piece in a second direction crossing the first direction.

4. The skin expansion apparatus of claim 3, wherein
each of the clamp includes:
an upper plate to contact with an upper surface of the skin piece;
a lower plate to contact with a lower surface of the skin piece; and
at least one needle protruding from at least one of the upper plate and the lower plate and penetrating through the skin piece.

5. The skin expansion apparatus of claim 4, wherein the lower plate have a guide hole to accommodate the needle.

6. The skin expansion apparatus of claim 4, further comprising:
a detachable clamp module provided in the chamber; and
a clamp connection bar connected to the clamp mode and each of the clamp.

7. The skin expansion apparatus of claim 6, further comprising:
a controller configured to control an expansion direction of the skin piece and an expansion time of the skin piece and to change a position of at least one of the clamp module and the clamp connection bar.

8. The skin expansion apparatus of claim 1, further comprising:
an environment controller configured to control a temperature of the chamber.

9. The skin expansion apparatus of claim 8, wherein the chamber has a bottom part and a side wall part connected to the bottom part, and the environment controller includes a water channel within the side wall part through which warm water moves.

10. The skin expansion apparatus of claim 9, wherein the environment controller further includes:
an inlet to provide the warm water to the water channel; and
an outlet to drain the warm water from the water channel.

11. The skin expansion apparatus of claim 10, further comprising a valve provided at at least one of the inlet and the outlet.

12. A method for expanding skin by using a skin expansion apparatus including a chamber and at least two clamps provided in the chamber, the method comprising:
fixing a skin piece within the chamber accommodating a culture medium with the clamps; and
pulling the skin piece in an outward direction with the clamps in a state in which the culture medium is put so as to expand the skin piece.

13. The method of claim 12, wherein the skin expansion apparatus further includes a operating controller configured to control an expansion direction of the skin piece and an expansion time of the skin piece.

14. The method of claim 13, wherein the pulling the skin piece is performed in one of a manual mode in which an expansion time of the skin piece is selected by a user and an automatic mode in which an expansion time of the skin piece is selected according to a preset value.

15. The method of claim 14, wherein the skin expansion apparatus includes a detachable clamp module provided in the chamber and a clamp connection bar connected to the clamp module and each of the clamps, and positions of the clamp module and the clamp connection bar are changed when the manual mode and the automatic mode are selected.

16. The method of claim 12, further comprising:
replacing the culture medium after the pulling the skin piece.

17. The method of claim 12, wherein a temperature of the chamber is maintained as a temperature of a human body when the skin expanding step is performed.
